SEQUENCE LISTING (4 sequences)

SEQ ID NO: 1 - protein, 309 aa, E. Coli
LDTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGCPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSKK

SEQ ID NO: 2 - protein, 283 aa, E. Coli
DNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYV

SEQ ID NO: 3 - protein, 243 aa, E. Coli
DNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLN

SEQ ID NO: 4 - protein, 185 aa, E. Coli
VVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKN

(19) United States
(12) Patent Application Publication
(10) Pub. No.: US 2011/0229415 A1
(43) Pub. Date: Sep. 22, 2011

(54) SEMI-SYNTHETIC ANTIBODIES AS RECOGNITION ELEMENTS

(76) Inventors: Sylvia Daunert, Coral Gables, FL (US); Leonidas G. Bachas, Coral Gables, FL (US); Boyd Haley, Nicholasville, KY (US); Smita Joel, Miami, FL (US)

(21) Appl. No.: 12/672,397

(22) PCT Filed: Aug. 6, 2008
(86) PCT No.: PCT/US08/72316

§ 371 Date: May 24, 2011

Related U.S. Application Data
(60) Provisional application No. 60/954,266, filed on Aug. 6, 2007.

(51) Int. Cl. A61K 49/00; C07K 16/00; G01N 33/53; A61B 5/1459

(52) U.S. Cl. 424/9.6; 530/391.3; 436/501; 600/317

(57) ABSTRACT

The presently-disclosed subject matter is directed to biosensors for detecting molecules of interest, and systems and methods for using same. The biosensors include an antibody and a probe covalently-linked to the antibody. The antibody has an antigen-binding site that selectively binds the molecule of interest and a purine-binding site, which is at a location distinct from that of the antigen-binding site. The probe includes a purine molecule, which is covalently bound at the purine-binding site to the antibody, and a label linked to the purine molecule. Upon binding of the molecule of interest to the biosensor antigen-binding site, the biosensor undergoes a conformational change, which detectably alters a signal of the label such that the molecule of interest can be detected.

### RELATED APPLICATIONS

This application claims priority from U.S. Provisional Application Ser. No. 60/954,266 filed Aug. 6, 2007. The entire disclosure of each application is incorporated herein by this reference.

### TECHNICAL FIELD

The presently-disclosed subject matter relates to methods and compositions for detection of molecules of interest.

### BACKGROUND

There are many changes that take place in the body of a subject under a variety of environmental and disease conditions. During these changes, electrolyte levels are modified, protein expression levels change in biological fluids, etc. Therefore, monitoring the levels of biomarkers in biological fluids, like blood, plasma and saliva for example, during changing environmental conditions or when a disease process is suspected is of great importance. Accordingly, there is a need to be able to detect certain molecules of interest, especially in a continuous manner. Further, it would be desirable to continuously detect these certain molecules in vivo.

Many biomarkers are present at concentrations that can be detected using antibody-based technologies. A number of antibodies specific for a wide variety of biomarkers are commercially available from a variety of sources including Abcam (e.g., osteocalcin, IL-1b, catecholamines), AbD Serotec (e.g., DHEAS, catecholamines), GeneTex (e.g., C-reactive protein), Sigma-Aldrich (e.g., cortisol, catecholamines), and others. In addition, antibodies can be generated against antigens of interest using standard laboratory techniques generally known to those of skill in the art. Likewise, for detection of microbial infection antibodies against cell surface antigens of the microbes are available or can be generated. However, traditional antibody-based immunoassays for detecting biomarkers of interest require that discrete biological samples be removed from the subject and then biomarkers from the samples immobilized to a substrate (e.g., directly or via selective binding to substrate-immobilized antibodies) in order to be detected. A separate labeling reaction with additional reagents is often required as well. These techniques therefore do not allow for continuous monitoring of biomarkers of interest and cannot be readily implemented as in vivo detection tools.

Accordingly, there remains a need in the art for sensors for detecting molecules of interest and a method of use which satisfactorily addresses the need of continuously detecting molecules of interest in vivo.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

In some embodiments of the presently-disclosed subject matter, a biosensor for detecting a molecule of interest is provided. In some embodiments, the biosensor comprises a probe, including a purine molecule and a fluorophore bound to the purine molecule; and an antibody, including an antigen-binding site that selectively binds the molecule of interest and a purine-binding site. The probe is covalently bound at the purine molecule to the purine-binding site. Binding of the molecule of interest to the antibody causes a conformational change in the antibody, which detectably alters a fluorescence intensity of the fluorophore, such that the molecule of interest can be detected.

In other embodiments of the presently-disclosed subject matter, a system for continuous in vivo detection of a molecule of interest in a body of a subject is provided. In some embodiments, the system comprises a detection and data collection device and a biosensor capable of recognizing the molecule of interest. The biosensor is operably connected with the data and collection device and includes a probe having a purine molecule and a fluorophore bound to the purine molecule and an antibody having an antigen-binding site that selectively binds the molecule of interest and a purine-binding site. The probe is covalently bound at the purine molecule to the purine-binding site. Binding of the molecule of interest to the antibody causes a conformational change in the antibody, which detectably alters a fluorescence intensity of the fluorophore such that the molecule of interest can be detected.

In still other embodiments of the presently-disclosed subject matter, a method for detecting a molecule of interest is provided. In some embodiments, the method comprises contacting a biosensor with a sample comprising the molecule of interest. The biosensor can be a biosensor as disclosed herein. The method further comprises binding the molecule of interest to the antibody, thereby resulting in a conformational change in the antibody, which detectably alters a fluorescence intensity of the fluorophore. The method further comprises detecting as a signal the altered fluorescence intensity of the fluorophore and then collecting and displaying the signal with a detection and data collection device, to thereby detect the molecule of interest. In some embodiments, the molecule of interest is continuously detected. In some embodiments, the molecule of interest is within an in vitro sample and in other embodiments the molecule of interest is within a blood stream in a body of a subject.

In some embodiments, the purine molecule is a nucleotide or a nucleoside, such as an adenine or a guanine. In some embodiments, the purine molecule is an ATP analog or a GTP analog, such as for example 2-azido ATP or 8-azido ATP. In some embodiments, the antibody is a monoclonal antibody. In some embodiments, the molecule of interest is Interleukin 6 (IL-6) or osteonectin.

In some embodiments, the fluorophore is an Alexa Fluor dye. The fluorescence intensity of the fluorophore can be correlated to the concentration of the molecule of interest in a sample. In some embodiments, the probe is a compound of Formula II:

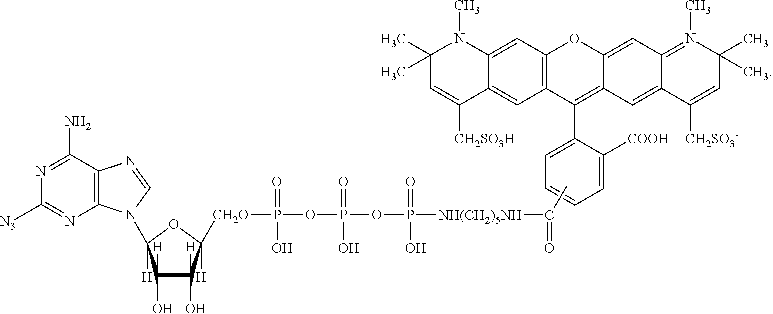

In some embodiments, the purine-binding site is located within the variable domain of the antibody and at a location distinct from that of the antigen-binding site. In some embodiments, the purine-binding site comprises invariant amino acid residues within the variable domain.

### DESCRIPTION OF THE DRAWINGS

FIG. 1 shows a schematic representation of a molecule of interest (analyte) binding to a biosensor disclosed herein comprising an antibody with an antigen-binding site (oval) and a probe (ATP-fluorophore) bound at a purine-binding site (rectangle) on the antibody. Upon binding at the antigen-binding site by the analyte, a conformational change occurs on the antibody, measurably altering a signal emitted by the biosensor (fluorescence), thereby allowing for detection of the analyte.

FIG. 2a shows the chemical structure of a radioactive probe [γ³²P] 2-N₃ATP for labeling of antibodies, which in this exemplary embodiment is the mouse anti-human IL-6 antibody.

FIG. 2b shows the chemical structure of a radioactive probe [γ-³²P] 8-N₃ATP for labeling of antibodies, which in this exemplary embodiment is the mouse anti-human IL-6 antibody.

FIG. 2c shows an autoradiogram of an antibody labeled with the photoreactive probes [γ-³²P] 2-N₃ATP (A) and [γ-³²P] 8-N₃ATP (B). Lanes 1, 4; 2, 5; and 3, 6 correspond to the use of 5 μl, 10 μl and 15 μl of labeled antibody, respectively.

FIG. 2d is a graph showing normalized data obtained from the autoradiogram of antibody labeled with the photoreactive probes shown in FIG. 1c.

FIG. 2e is a graph showing saturation of photolabeling of antibody with [γ-³²P] 2-N₃ATP.

FIG. 3a shows the chemical structure of a fluorescent nucleotide probe: 2-N₃ATP-Alexa Fluor 594 cadaverine.

FIG. 3b is a graph showing the affect of analyte on the fluorescence intensity of the probe as a dose response curve for IL-6. Data are the average of ±one standard deviation (n=3). All error bars are less than 10%.

FIG. 4 is a graph showing the determination of the apparent K_{D} of the mouse anti-human IL-6 monoclonal antibody for 2-N₃ATP[γ]-ALEXA FLUOR® 594 cadaverine.

FIG. 5 is a graph showing a calibration curve for monoclonal antibody against human Osteonectin.

FIG. 6 shows labeling of mAb against Osteonectin antibody with radioactive probes [γ³²P] 2N₃ATP versus [γ³²P] 8N₃ATP. Lanes A and D have 5 B and E have 10 μL, and C and F have 15 μL of the labeled antibody. [γ³²P] 2N₃ATP labels the antibody better than [γ³²P] 8N₃ATP.

FIG. 7 is a graph showing an association curve for Osteonectin.

FIG. 8 is a graph showing a dose response curve for Osteonectin.

FIG. 9 is a graph showing a selectivity study of a mAb against Osteonectin for Osteonectin and Interleukin 6.

FIG. 10A is a ribbon diagram of an anti-cortisol antibody showing the nucleoside binding site.

FIG. 10B is a space-filled structure of the antibody's binding pocket showing the amino acids that interact with the nucleoside.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

### DEFINITIONS

While the following terms are believed to be well understood by one of ordinary skill in the art, the following definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the terms “about,” “approximate,” and “approximately,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the terms “label” and “labeled” refer to the attachment of a moiety, capable of detection by spectroscopic, radiologic, or other methods, to a probe molecule. Thus, the terms “label” or “labeled” refer to incorporation or attachment, optionally covalently or non-covalently, of a detectable marker into a molecule, such as a probe. Various methods of labeling molecules are known in the art and can be used. Specific examples are described herein. Fluorescent probes that can be utilized include, but are not limited to fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, 3, 3.5, 5, 5.5, and 7; phycoerythrin; phycoerythrin-Cy conjugates; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins; coumarin derivatives (e.g., hydroxycoumarin, aminocoumarin, and methoxycoumarin); pyrenes; pyridyloxazole derivatives; dapoxyl dyes; Cascade Blue and Yellow dyes; benzofuran isothiocyanates; sodium tetrafluorophenols; 4,4-difluoro-4-bora-3a,4a-diaza-s-indacene; ALEXA FLUOR® dyes (e.g., 350, 430, 488, 532, 546, 555, 568, 594, 633, 647, 660, 680, 700, and 750); green fluorescent protein; yellow fluorescent protein; and fruit fluorescent proteins. The peak excitation and emission wavelengths will vary for these compounds and selection of a particular fluorescent probe for a particular application can be made in part based on excitation and/or emission wavelengths.

The terms “polypeptide,” “protein,” and “peptide,” which are used interchangeably herein, refer to a polymer of the 20 protein amino acids, including modified amino acids (e.g., phosphorylated, glycated, etc.) and amino acid analogs, regardless of size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein”, “polypeptide” and “peptide” are used interchangeably herein when referring to a gene product and fragments thereof. Thus, exemplary polypeptides include gene products, naturally occurring proteins, homologs, orthologs, paralogs, fragments and other equivalents, variants, fragments, and analogs of the foregoing. In some embodiments, the term polypeptide includes a conservatively substituted variant.

The term “conservatively substituted variant” refers to a peptide comprising an amino acid residue sequence that differs from a reference peptide by one or more conservative amino acid substitution, and maintains some or all of the activity of the reference peptide as described herein. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another. The phrase “conservatively substituted variant” also includes peptides wherein a residue is replaced with a chemically derivatized residue, provided that the resulting peptide maintains some or all of the activity of the reference peptide as described herein.

The terms “polypeptide fragment” or “fragment”, when used in reference to a polypeptide, refers to a polypeptide in which amino acid residues are absent as compared to the full-length polypeptide itself, but where the remaining amino acid sequence is usually identical to the corresponding positions in the reference polypeptide. Such deletions can occur at the amino-terminus or carboxy-terminus of the reference polypeptide, or alternatively both. A fragment can retain one or more of the biological activities of the reference polypeptide. In some embodiments, a fragment can comprise a domain or feature, and optionally additional amino acids on one or both sides of the domain or feature, which additional amino acids can number from 5, 10, 15, 20, or more residues.

The term “purine molecule,” as used herein refers to heterocyclic aromatic organic compounds having a pyrimidine ring fused to an imidazole ring. “Purine molecule,” as the term is used herein is inclusive of nucleosides and nucleotides, such as adenine (e.g., ATP) and guanine (e.g., GTP).

As used herein, the term “selectively bind” refers to an interaction between a molecule of interest, e.g., IL-6 or osteonectin, and a binding site of a polypeptide molecule (e.g., an antigen-binding site of an antibody). In some embodiments, the interaction between a molecule of interest, and the binding site, can be identified as “selective” if: the equilibrium dissociation constant (K_{d}) for the compound of interest is less than the K_{d} of other molecules present in the sample; The equilibrium inhibitor dissociation constant (Kᵢ) for the molecule of interest is less than the Ki of other molecules present in the sample; or the effective concentration at which binding of the molecule of interest gives 50% of the maximum response (EC₅₀) is less than the EC₅₀ of other molecules present in the sample. In some embodiments, the interaction between a molecule of interest, and the binding site, can be identified as “selective” when the equilibrium dissociation constant (K_{d}) is less than about 100 nM, 75 nM, 50 nM, 25 nM, 20 nM, 10 nM, 5 nM, or 2 nM.

As used herein, the term “detect” means to determine quantitatively and/or qualitatively. In particular, with regard to the presently-disclosed subject matter, a molecule of interest is detected when the molecule binds to an antigen-binding site of a biosensor disclosed herein, causing a conformational change in the biosensor, which results in a detectable alteration in a signal emitted by a label associated with the biosensor. The change in signal is correlated with the presence of the molecule of interest in the sample, and thereby the molecule is detected. In addition, if desired, the signal change can be utilized to quantitatively determine an amount of the molecule of interest in the sample.

As used herein, the term “amount” means a quantitative and/or qualitative value, and can refer to the presence or absence of.

### Biosensors

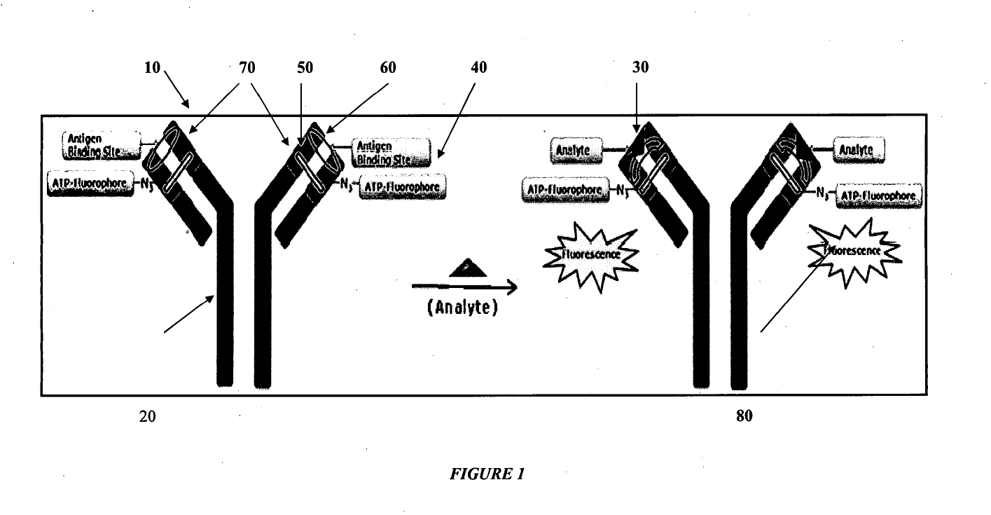

The characteristic property of antibodies to bind strongly and selectively to antigens make them ideal analytical tools for the development of biosensors for a wide variety of analytes. The presently-disclosed subject matter provides a universal method to develop novel biosensors for various molecules of interest, including biomarkers indicative of disease, changes in physiology, and detection of microorganisms. As shown in FIG. 1, the presently-disclosed novel biosensors 10 are based on an antibody 20 having binding specificity for a molecule of interest 30 (e.g., analyte or antigen) and conjugated (e.g., via photolabeling) with a probe 40 comprising a purine molecule (e.g., ATP) and a label (e.g., a fluorophore) that emits a signal upon binding of the antibody to the molecule of interest. A novel feature of the present biosensors is that the probe is bound to the antibody at an unconventional purine-binding site 50 (distinct from the canonical antigen-binding site 60) that is highly conserved among at least the IgG and IgM classes of antibodies. For example, trypsinization followed by mass spectrometry identified three amino acids, Trp103 (from the heavy chain), Tyr36 (from the light chain), and Asp101 (from the heavy chain) of the anti-RNA antibody 1MRC (described in Pokkuluri et al., 1994) as being modified by the photoaffinity probe. As disclosed in detail in Example 3, this area of the variable regions of antibody light and heavy chains is highly conserved across a wide variety of antibodies with varying antigen specificities, particularly at these three residues where the probe binds. As such, this conserved purine-binding site can be utilized for attachment of purine-based labeled probes to most if not all antibodies to generate highly specific and rugged biosensors tailored to detect a particular molecule of interest.

Nucleotide photoaffinity probes have been utilized successfully for the characterization of nucleotide binding sites in proteins (Haley, 1991). With reference again to FIG. 1 and as disclosed above, a novel purine-binding site 50 is present in the variable immunoglobulin (Ig) domain 70 formed by invariant residues of both light and heavy chains of Ig, which binds purine-containing nucleotide photoaffinity probes with high affinity (see, e.g., U.S. Pat. Nos. 5,800,991 and 5,596,081 and Rajgopalan et al., 1996, each of which is incorporated herein by reference). The purine ring is held via base stacking interactions within the novel site while the phosphate groups and the ribose ring (present if the probe 40 comprises a nucleotide) are exposed to the surface of the molecule allowing a number of labels to be tethered to the antibody 20 via the purine molecule (Rajgopalan et al., 1996; Pavlinkova et al., 1997). Docking to these unconventional sites does not interfere with the antigen binding (Pavlinkova et al., 1997). However as now surprisingly determined and as capitalized on by the present novel biosensors, binding of the antigen 30 to the antigen-binding site 60 of the antibody variable region 70 triggers conformational changes at the purine-binding site 50. As shown in FIG. 1, the conformational change induces a detectable change in a signal 80 emitted by the label portion of the probe 40 bound to the antibody 20. The change in signal 80 is correlated with the binding, and therefore presence of, the molecule of interest. Thus, a label portion of the probe is selected based in part on its emitted signal 80 being detectably altered upon the conformational change in the antibody 20. Fluorophores are one example of labels that generally can meet these criteria for the novel biosensors of the presently-disclosed subject matter. For example, and without wishing to be bound by any particular theory of operation, with reference to fluorophores, a conformational change in the antibody 20 can result in a change in fluorescent signal, including for example a shift in the wavelength of emission energy, a change in fluorescence intensity, and/or a change in fluorescence lifetimes. The change in signal can be measured and then correlated to binding of the molecule of interest.

As such, the presently-disclosed subject matter provides a reagentless highly sensitive and selective universal biosensing system based on an antibody conjugated with a labeled purine probe. Selective binding of the molecule of interest to the antigen-binding site of the antibody produces a conformational change in the antibody that generates a detectable signal from the label, which allows for detection of the molecule of interest. This novel biosensor system is universal as it can be used for the detection of a wide variety of molecules of interest depending only on the availability of an antibody for binding to the molecule. Further, as it does not require additional reagents or fixation of the molecule of interest to a substrate prior to detection, it can be used to continuously detect molecules of interest, including in an in vivo setting.

Thus, in some embodiments of the presently-disclosed subject matter, a biosensor for detecting a molecule of interest is provided. The biosensor can comprise an antibody and a probe covalently bound to the antibody. The probe can include a purine molecule and a label bound to the purine molecule. In some embodiments, the label can be a fluorophore. The antibody includes an antigen-binding site that selectively binds the molecule of interest, as well as a purine-binding site distinct and separate from the antigen-binding site. The probe can be covalently-bound at the purine molecule to the antibody at the antibody purine-binding site.

Fluorophores are functional groups in a molecule that can absorb energy of a specific wavelength and re-emit energy at a different (but equally specific) wavelength, resulting in detectable fluorescence of the molecule. The amount and wavelength of the emitted energy depend on both the fluorophore and the chemical environment of the fluorophore. One exemplary fluorophore is fluorescein isothiocyanate, a reactive derivative of fluorescein, which has been one of the most common fluorophores utilized for a variety of applications. Other historically common fluorophores are derivatives of rhodamine, coumarin and cyanine. Fluorophores also include newer molecules, including the ALEXA FLUORS® (Invitrogen, Carlsbad, Calif., U.S.A.). In some embodiments, the probe fluorophore is an ALEXA FLUOR® dye, such as for example ALEXA FLUOR® 594 cadaverine. The chemical structure of ALEXA FLUOR® 594 cadaverine is set forth in Formula I. Other fluorophores of interest, for example, include OREGON GREEN® 488 cadaverine and TEXAS RED® cadaverine.

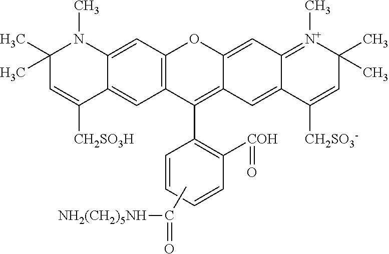

Any antibody which effectively binds purine molecules at a site that is different than the canonical binding site is within the scope of the presently-disclosed subject matter. This includes by way of example, polyclonal and monoclonal antibodies, recombinant antibodies, chimeric antibodies, humanized antibodies, bispecific antibodies, single chain antibodies, antibodies from different species (e.g., mouse, goat, rabbit, human, rat, bovine, etc.), anti-idiotypic antibodies, antibodies of different isotype (IgG, IgM, IgE, IgA, etc.), as well as fragments and derivatives thereof (e.g., (Fab)₂ Fab, Fv, Fab, 2(Fab), Fab′, (Fab′)₂ fragments), so long as the antibodies, fragments, or derivatives include an antigen-binding site that selectively binds a molecule of interest (i.e., a “canonical site”) and a purine-binding site distinct from the antigen-binding site (i.e., a “non-canonical site”).

A “purine-binding site” is a region within the antibody that binds purine molecules. It has been determined that antibodies can comprise one or more regions that have high binding affinity for purine molecules, and these molecules will readily attach to antibodies at these regions, even under physiological conditions. In some embodiments, the purine-binding regions are found within the variable domain of the antibody. In particular embodiments, the purine-binding regions comprise invariant amino acid residues within the variable heavy (V_{H}) and variable light (V_{L}) peptide chains of the antibody. In some embodiments, the probes can be covalently bound to the antibodies at the purine-binding sites by a photoactivated chemical reaction. For example, in some embodiments a relatively short photolysis reaction using ultraviolet light can be applied to the antibodies and bound probes in solution, resulting in covalent binding of the probes at the purine molecules (e.g., via an azido group) to the purine-binding region of the antibodies.

One novel and advantageous feature of the presently-disclosed biosensors is that upon binding of the molecule of interest within the antigen-binding site of the antibody, the antibody undergoes a conformational change, which alters a signal of the label, such as for example a fluorescence intensity, a shift in emission wavelength, and/or a change in fluorescence lifetime of a fluorophore label. That is, changes in the conformation of the antibody result in a change in the environment around the fluorophore, resulting in a change in the signal. Therefore, environment-sensitive fluorophores can be selected which show a change in the fluorescence intensity, emission wavelength, and/or a fluorescence lifetime based on the change in the conformation of the antibody and resultant change in local environment of the fluorophore. The change in fluorescence signal is detectable and directly correlative to the presence of the molecule of interest in a sample. The fluorescence signal can be correlated to the concentration of the molecule of interest in the sample. As such, the molecule of interest can be not only detected, but also quantitated using the biosensors disclosed herein. In addition, utilizing the presently-disclosed sensors precludes the need to separate the antigen-bound antibody from the unbound antibody, a major advantage over conventional immunoassay technologies (e.g., ELISA, fluorescence immunoassay, etc.), as data can be obtained continuously, as well as in vivo, such as for example, when the biosensor is incorporated as a component of a system for in vivo use comprising a data detection and collection device disclosed herein. Further, since binding of the molecule of interest to the antibody portion of the sensor results in a conformational change to the antibody that directly results in a change in fluorescence of the label, additional reagents for detecting signal are not required. As such, the present novel biosensors afford the advantage of use within a “reagentless” system for detection, which can further allow for use of the present biosensors in vivo.

In exemplary embodiments, the molecule of interest is Interleukin 6 (IL-6) or osteonectin and the biosensor antibody selectively binds and detects IL-6 or osteonectin, respectively. In some embodiments, the antibody is a monoclonal antibody that selectively binds the molecule of interest. In some embodiments, the biosensor comprises an antibody that specifically binds IL-6 or osteonectin and a probe covalently bound to the purine-binding site of the antibody and comprising a purine molecule covalently bound to an ALEXA FLUOR® 594 cadaverine fluorophore, for example. Other examples of molecules of interest include, but are not limited to, other interleukins in the family of cytokines, hormones, drugs, drugs of abuse, cardiac markers, vitamins, cancer biomarkers, inflammation markers, and peptides and polysaccharides specific for microorganisms.

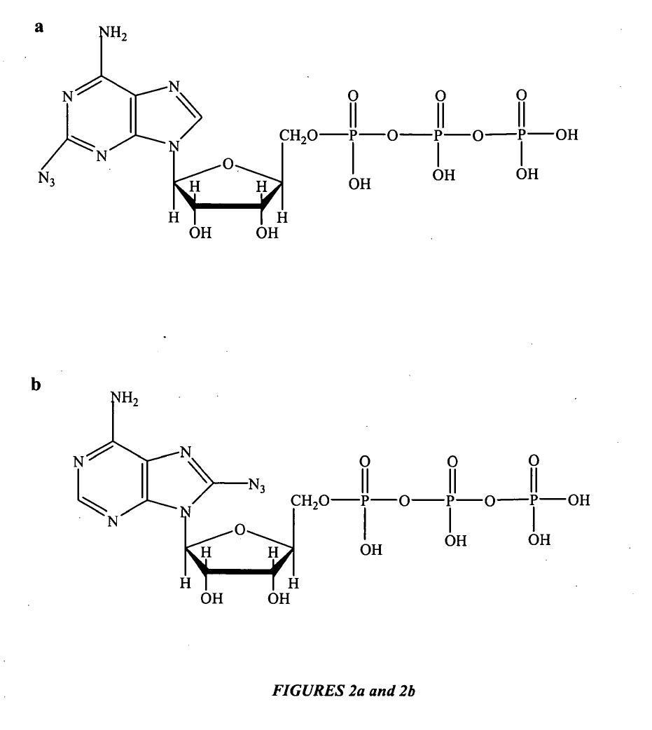

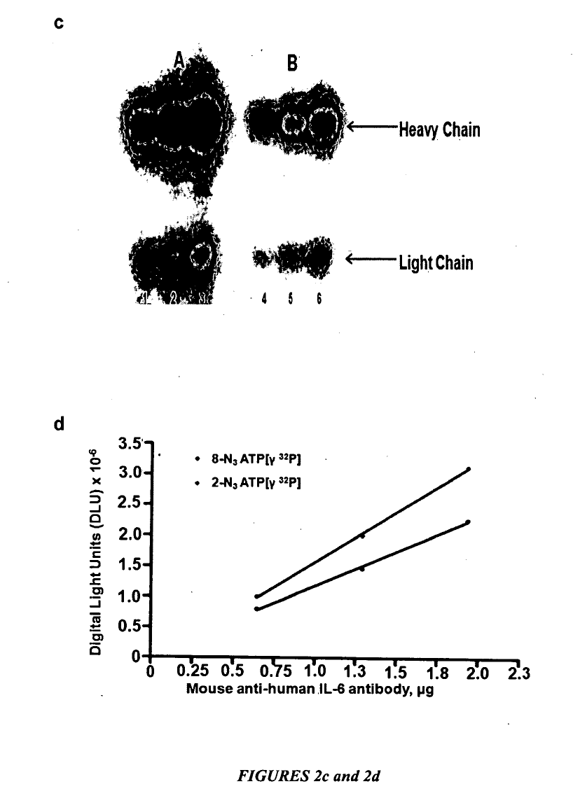

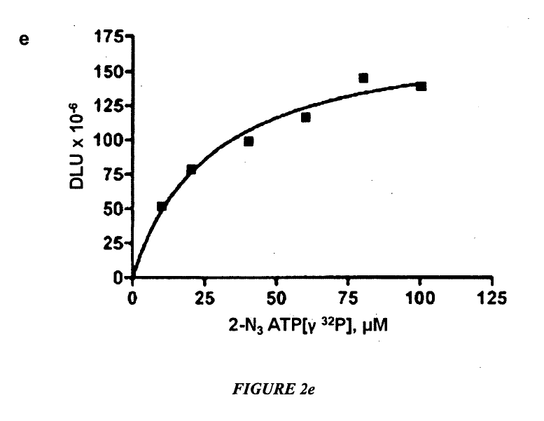

In some embodiments, the purine molecule component of the probe can be an adenine (e.g., ATP) or guanine (e.g., GTP), or an analog or derivative thereof. Exemplary purine derivatives include azido-purines, such as for example azido-adenines and azido-guanines. Additional exemplary purine molecules that can be utilized with the probes disclosed herein include, but are not limited to: 2-azido or 2-azidoadenylyl(2′-5′)2-azidoadenylyl(2′-5′)2-azidoadenosine; 2-azido or 8-azidoadadenosine; 8-azidoadenylyl(2′-5′)-8-azidoadenylyl(2′-5′)8-azidoadenosine; 8-azidoadenylyl(2′-5′)8-azidoadenylyl(2′-5′)8-azidoadenylyl-(2′-5′)8-azido adenosine; 2,8-diazidoadenylyl(2′-5′)2,8-diazidoadenylyl(2′-5′)2,8-diazido-adenosine; 2,8-diazidoadenylyl(2′-5′)2,8-diazidoadenylyl(2′-5′)2,8-diazidoadenylyl(2′-5′)2,8-diazidoadenosine; also oligomers of AMP and a single azidoadenylyl species, such as, for example: 2-azidoadenylyl(2′-5′)2-(2′-5′)adenosine; adenylyl(2′-5′)8-azido-adenylyl(2′-5′)8-azidoadenosine; also oligomers containing more than one azidoadenylyl species, such as, for example: 2-azido-adenylyl(2′-5′)8-azidoadenylyl(2′-5′)2-azidoadenosine; also oligomers resulting from any combination of the monomers AMP, 2-azido-AMP, 8-azido-AMP and/or 2,8-diazido-AMP, provided that at least one such monomer incorporated into the oligomer is an azido-AMP species. In some particular embodiments, the purine molecule is 2-azido ATP (FIG. 2a) or 8-azido ATP (FIG. 2b).). In some particular embodiments, molecules with structural similarity to purines (e.g., aromatic compounds) can be substituted for 2-azido ATP. In some particular embodiments, the probe comprises 2-azido ATP covalently linked to ALEXA FLUOR® 594 cadaverine. The chemical structure for this particular embodiment of the probe is set forth in Formula II.

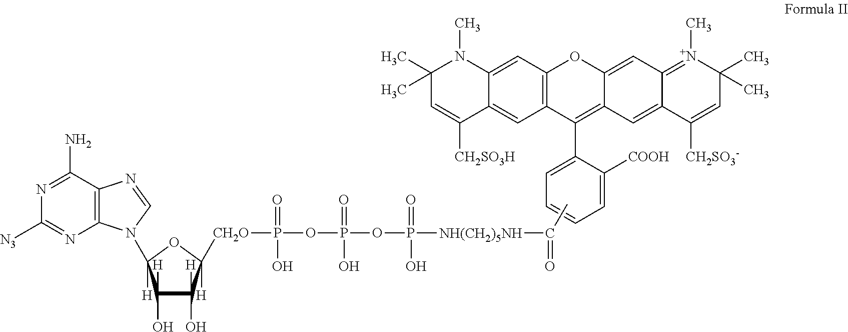

### System for Detecting Molecules of Interest

The biosensors of the presently-disclosed subject matter can be utilized in a number of different capacities in order to detect molecules of interest, both in vitro and in vivo. As one non-limiting example, the biosensor can be coupled with a detection and data collection device along with a platform for presenting the biosensor to the biological sample being tested for a molecule of interest.

In some embodiments, the system for detecting molecules of interest can comprise a biosensor as disclosed herein coupled with a catheter comprising an optical fiber for continuous in vivo detection of a molecule of interest in a body of a subject. When the molecule of interest is bound by the antibody, the biosensor emits a signal that is transmitted by the optical fiber to the detection and data collection device. A label associated with the biosensor generates the signal. The label can be, for example, a fluorescent label or an electrochemical label. A label can be selected based on the selected antibody portion of the biosensor, the probe section of the biosensor, and/or the molecule of interest. For example, in some embodiments, the label can comprise a fluorophore, such as for example ALEXA FLUOR® 594 cadaverine.

Any known catheter suitable for implantation in a body can be utilized with the biosensors disclosed herein, including but not limited to catheter systems disclosed in U.S. Patent Application No. XX/XXX,XXX to Daunert et al. entitled “DEVICE FOR DETECTION OF MOLECULES OF INTEREST,” claiming priority to U.S. Provisional Application Ser. Nos. 60/954,269 and 60/954,348, and filed on Aug. 6, 2008 (hereinafter the “Daunert et al. Application), which is incorporated herein by reference in its entirety. In these embodiments, the detection and data collection device can be any device known in the art that can receive a signal transmitted from the biosensor by an optical fiber system, which converts the light signal from the optical fiber to an electrical signal. An example of such a data collection device is an Edwards Lifesciences Vigilance II monitor (Model Number VIG2).

In addition to coupling with catheter systems, the biosensors disclosed herein can also be utilized in several other systems for detection of molecules of interest. For example, the biosensors disclosed herein can be employed for the development of sensing systems on non-catheter platform systems. For example, the biosensors disclosed herein can be used to quantitate biomarker levels on both microtiter plate and miniaturized microfluidics platforms, which are popular in high-throughput screening, clinical laboratory practice, and in the development of point-of-care diagnostic equipment. Additionally, the presently-disclosed biosensors can be immobilized on affordable and robust paper strips, whose visible color change would correlate to biomarker levels. These paper strips would be a practical and competitive option for rapid clinical or patient self-monitoring of biomarker levels. Moreover, the dynamic range of specific molecule detection by the biosensors of the presently-disclosed subject matter may permit salivary analysis of different biomarker levels.

In addition, the biosensors disclosed herein can be contained within a hydrogel, such as part of a catheter system as disclosed for example in the ‘Daunert et al. Application, but can in a hydrogel for the development and improvement implantable contact lens biosensors, including biosensors coupled with drug delivery devices. As an example of a potential use in implantable drug delivery devices, the change in fluorescent or electrical signal caused by a biomarker binding to the biosensor could be translated to the opening and closing of a reversible drug-containing reservoir. In this manner biomarker levels can be both monitored and corrected in subjects. Additionally, current contact lens sensors for glucose have a sensing plastic chip incorporated into the regular corrective lens. This plastic chip changes colors via holographic sensing methods and boron-containing fluorophores. These color changes are visible to the wearer, with different colors corresponding to different glucose levels in tears, thus alerting the patient if insulin is needed. The present biosensors could be comparably incorporated into such a system to provide visual notice to a subject of an unsafe biomarker level requiring attention. Utilizing the present biomarkers with hydrogels in these systems provides additional advantages as hydrogels are more water and oxygen permeable than the plastic chips that are currently used, and this permeability is quite important for both the comfort and long term optical health of the contact lens wearer.

### Method of Detecting Molecules of Interest

An exemplary method for detection of a molecule of interest includes initially contacting a biosensor with a sample comprising the molecule of interest. The biosensor can include a probe, including a purine molecule and a label (e.g., a fluorophore) bound to the purine molecule, and an antibody. The antibody can include an antigen-binding site that selectively binds the molecule of interest and a purine-binding site. The probe can be covalently bound at the purine molecule to the purine-binding site. The method further includes binding the molecule of interest to the antibody, thereby resulting in a conformational change in the antibody, which detectably alters a signal (e.g., fluorescence intensity, emission wavelength, and/or fluorescence lifetime) of the label. Next, the method includes detecting this signal and then collecting and displaying the signal with a detection and data collection device, to thereby detect the molecule of interest. In some embodiments, the molecule of interest is continuously detected.

In some embodiments, the molecule is within a sample. The sample can be a blood stream or body fluid in a body of a subject, in vivo, or can be an in vitro sample.

The presently-disclosed subject matter is further illustrated by the following specific but non-limiting examples. The following examples may include compilations of data that are representative of data gathered at various times during the course of development and experimentation related to the present invention.

### EXAMPLES

### Example 1

To demonstrate the feasibility of designing and utilizing the presently-disclosed biosensors for detecting a wide variety of molecules of interest, we utilized the highly sensitive and selective interaction of mouse anti-human monoclonal antibody against Interleukin 6 (IL-6) for molecular recognition. IL-6 is a multifunctional cytokine consisting of 185 amino acids (Barkenhoff et al., 1989) and secreted by T cells and macrophages. IL-6 promotes inflammatory events by activation of T cells, differentiation of B cells, and the induction of acute phase reactants by hepatocytes (Hirano et al. 1986; Hirano et al. 1986). Besides these activities, IL-6 also plays a protective role during disease, acting both as a pro- and anti-inflammatory cytokine (Jones et al., 2001). Elevated concentration of IL-6 in body fluids has been reported in various disease states, such as cardiac myxomas and cardiovascular diseases (Mendoza et al., 2001; Volpato et al., 2001; Luc et al., 2003).

To generate a biosensor, a novel purine-binding site of the antibody, located between the light and heavy chains for signal transduction, was targeted for attachment of a probe as disclosed herein comprising a purine molecule conjugated with a fluorophore. The antibody purine-binding site selectively binds the probe at the purine molecule. The probe can then be covalently bound to the antibody. The probe can be docked to the purine-binding site without interfering with analyte binding.

We labeled a mouse anti-human IL-6 monoclonal antibody with two radioactive probes, [γ-³²P]-2-N₃ATP (FIG. 2a) and [γ-³²P]-8-N₃ATP (FIG. 2b) to test their reactivity towards the antibody (see Example 1 Methods). Both light and heavy chains were labeled with the radioactive probe indicating the presence of a purine-binding site between the two chains (FIG. 2c). It was also determined that [γ-³²P]-2-N₃ATP labels the antibody better than [γ-³²P]-8-N₃ATP in this biosensor (FIG. 2d). Further to optimize the efficiency of labeling the antibody with the photoreactive probe, concentration of the probe required to label the antibody quantitatively was determined by incubating mouse anti-human IL-6 monoclonal antibody with increasing concentrations of [γ-³²P]-2-N₃ATP followed by photoactivation of the samples (see Example 1 Methods). The saturation of photolabeling was observed at ˜25 ΞM of the photoreactive [γ-³²P]-2-N₃ATP (FIG. 2e).

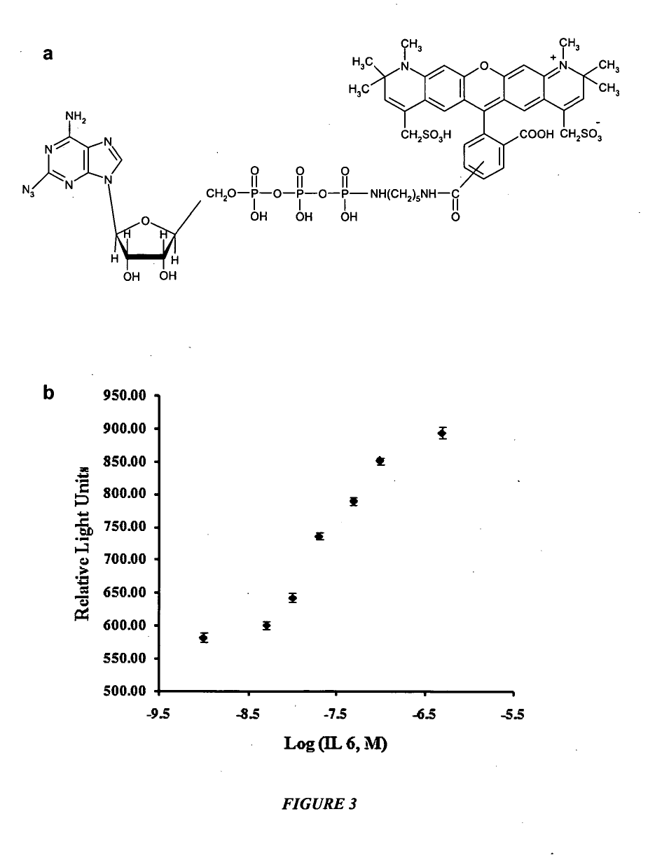

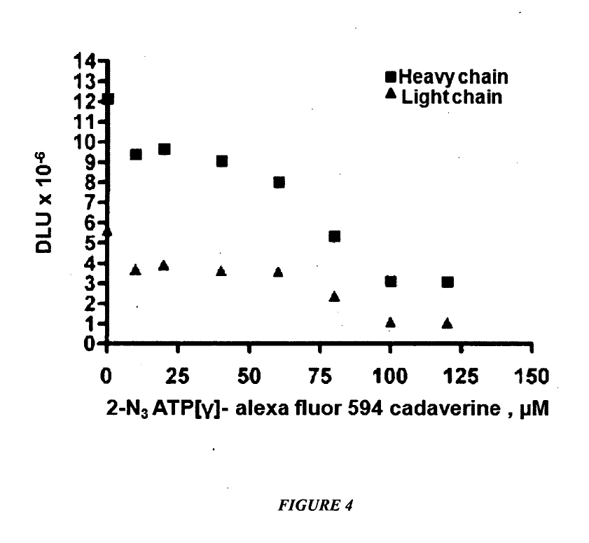

In an effort to develop an exemplary in vitro/in vivo biosensing system for IL-6, ALEXA FLUOR® 594 cadaverine, a fluorophore with high excitation and emission wavelengths (590 nm/617 nm, respectively) was selected to circumvent the interferences from the common fluorescent interferents present in some biological samples. The amine functional group of ALEXA FLUOR® 594 cadaverine was utilized for conjugation to 2-N₃ATP (see Example 1 Methods), as it was observed that 2-N₃ATP labels this IL-6 antibody with a greater efficiency as compared to 8-N₃ATP. The apparent K_{D} for 2-N₃ATP-Alexa Fluor 594 cadaverine (structure shown in FIG. 3a) was determined to be ˜17 μM (see Example 1 Methods and FIG. 4). The fluorophore labeled nucleotide was further conjugated to the mouse anti-human IL-6 monoclonal antibody at the novel site present between light and heavy chains of the variable region (see Example 1 Methods).

The effect of antigen (IL-6) binding on the fluorescence signal of the 2-N₃ATP-Alexa Fluor 594 cadaverine label attached to the antibody was evaluated (see Example 1 Methods for details). The antibody has an antigen-binding site for IL-6 in the variable region above the purine-binding site where the probe binds. Without wishing to be bound by theory, the binding of antigen to the variable region of antibody induces a change in the conformation of antibody resulting in a change in the signal of fluorophore attached to the purine-binding site, which is distinct from the antigen-binding site. It was observed that binding of IL-6 to the antibody resulted in an increase of the signal (e.g., fluorescence intensity) emitted by the fluorophore portion of the probe, which increased with the increase in the concentration of antigen (FIG. 3b). This increase in the intensity of fluorescence signal can be attributed to the change in the conformation of the variable region of antibody. Since the purine-binding site on the antibody where the fluorescent probe is docked is located below the antigen-binding site in the variable region of antibody, the conformational changes of the antibody binding site affect the purine-binding site region where the probe is bound, altering the fluorescence signal of the fluorophore.

We conclude that a fluorophore based purine probe can bind to the unconventional site (i.e., the purine-binding site) of an antibody without affecting the selective antigen binding capabilities of the antibody. Additionally, the present data demonstrate that binding of the antigen to the antibody portion of the biosensor induces conformational changes to the antibody, which can alter the fluorescence intensity of the fluorophore-based purine probe docked to the unconventional site of the antibody. The change in the fluorescence signal intensity of the label can be correlated to the concentration of molecule of interest in the sample.

It is anticipated that most, if not all, antibodies comprise a comparable purine-binding site in the variable region. Thus, the exemplary methodology demonstrated in the present example can be applied broadly to antibody-based biosensors specific for a wide variety of antigens. Further, a number of different reporter molecules including drugs, metal chelates, or peptides can be attached to the unconventional site of the antibodies without affecting the antigen binding ability of the antibody. Thus, the novel biosensor platform is suitable for detecting a great variety of molecules of interest, so long as an antibody that selectively binds the molecule of interest can be developed.

### Methods for Example 1

Selecting the photoaffinity probe. Two radioactive nucleotide probes, [γ-³²P]8-N₃ATP and [γ-³²P]2-N₃ATP were tested towards their reactivity with Mouse anti-Human IL-6 monoclonal antibody. For the labeling procedure 8 μg antibody was incubated with 300 μM of each probe in two different tubes in a final volume of 62 μl for 10 min on ice. The buffer used was 100 mM BTP at pH 4.0. After 10 min incubation the two tubes were photolyzed with a hand held UV lamp at 254 nm for 2 min and the reaction mixture was agitated in between to prevent local heating. The reaction was quenched by adding 12 μl of loading dye, PSM (5×) and heating it in a water bath for 5 min. The reaction products were then analyzed by SDS PAGE. ³²P incorporation was detected by autoradiography.

Saturation of labeling. 2 μg of Mouse anti-Human IL-6 monoclonal antibody was incubated with increasing concentrations of [γ-³²P]2-N₃ATP for 20 min on ice and photolyzed with a hand held lamp at 254 nm for 1 min 15 sec. The reaction was quenched by adding 5 μl of 0.25 mg/ml cysteine and analyzed by SDS/PAGE and autoradiography.

Conjugation of 2-N₃ATP to ALEXA FLUOR® 594 cadaverine. The conjugation of 2-N₃ATP (2-azido ATP) to the fluorophore was done at Affinity Labeling Technologies (ALT) Corporation (Lexington, Ky.). 1:1 ratio of 2-N₃ATP and ALEXA FLUOR® 594 cadaverine was added to 0.1 M EDC (1-ethyl-3-(3-dimethylaminopropyl) carbodiimide hydrochloride) and 5 mM sulfo-NHS (N-Hydroxysulfosuccinimide) and allowed to react at room temperature for 2 h. The conjugate was then purified using DEAE (diethylaminoethyl) cellulose column and triethylammonium carbonate (0-300 mM) as elution gradient.

Determination of K_{D} of the monoclonal antibody for 2-N₃ATP-fluorophore. Competitive labeling of the mouse anti-human IL-6 antibody with 2-N₃ATP [γ ³²P] and 2-N₃ATP-fluorophore was done. 30 μM 2-N₃ATP [γ ³²P] and 2 μg of the antibody was added to each 10 μM, 20 μM, 40 μM, 60 μM, 80 μM, 100 μM and 120 μM of 2-N₃ATP-fluorophore tube and incubated for 20 min on ice. The tubes were then photolyzed for 1 min 15 s. The reaction was quenched by adding 5 μl of 0.25 mg/ml cysteine and analyzed by SDS/PAGE and autoradiography.

Conjugation of the antibody to the 2-N₃ATP-fluorophore. For the conjugation of 2-N₃ATP—ALEXA FLUOR® 594 cadaverine to the antibody against IL-6, 4 μg of the antibody was incubated with 30 μM 2-N₃ATP labeled fluorophore in a final volume of 50 μl for 20 min, pH 4.0, in ice and in dark. The reaction was photolyzed with a hand held UV lamp at 254 nm for 4 mins. To quench the photolytic reaction cysteine (10 μl of 1 mg/ml) was added. The unbound fluorophore was separated from the antibody bound fluorophore by dialyzing against six changes over 24 hrs of 10 mM phosphate buffer at pH 7.0.

Fluorescence studies of the interaction of the labeled antibody with IL-6. The effect of the antigen binding on the fluorescence signal of the label attached to the unconventional site of the IL-6 antibody was studied by incubating 4 μg/ml of the labeled antibody with different concentrations of IL-6 for 30 min at room temperature in the dark. The sample solutions were prepared using 10 mM phosphate buffer, pH7.0. The fluorescence intensity of the samples (total volume 200 μL) was measured on a Cary Eclipse Spectrofluorometer.

### Example 2

The present example provides another exemplary biosensor that specifically detects Osteonectin. Similarly to the biosensor of Example 1, this biosensor comprises an antibody, which in this embodiment selectively binds Osteonectin, covalently bound with a probe at a novel purine-binding site of the antibody, located in the variable region between the light and heavy chains of the antibody. The probe portion of the biosensor comprises a purine molecule (2-azido ATP) conjugated with a fluorophore (ALEXA FLUOR® 594 cadaverine).

Osteonectin is an acidic, noncollagenous glycoprotein (Mr=32,700) in the bone that binds calcium. It is secreted by osteoblasts during bone formation, initiating mineralization and promoting mineral crystal formation. Osteonectin contains aspartic and glutamic acid rich domains besides NH₂-terminal domain and a cysteine-rich domain. Two forms of osteonectin, bone derived and platelet derived are known, of which bone osteonectin binds to collagen type I and hydroxyapatite, suggesting its importance in the regulation of bone mineralization. Studies show that Osteonectin is a salivary biomarker associated with periodontal disease as elevated concentration of osteonectin is found in the gingival cervical fluid of subjects with periodontal disease.

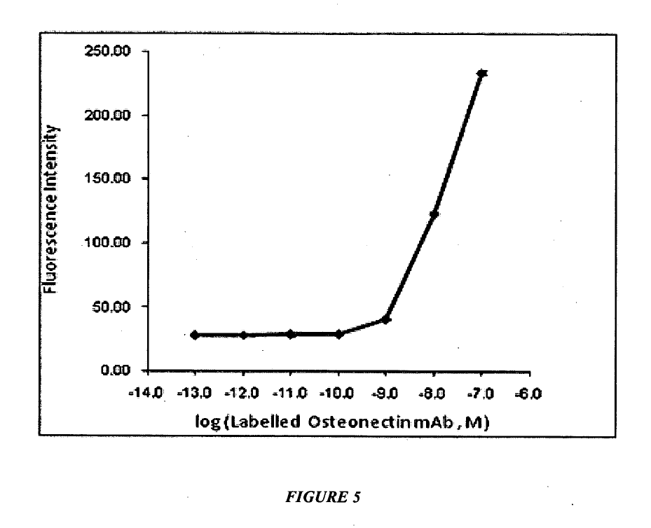

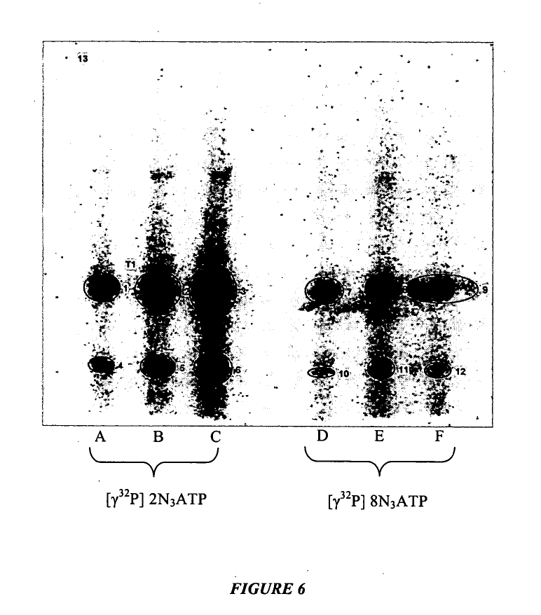

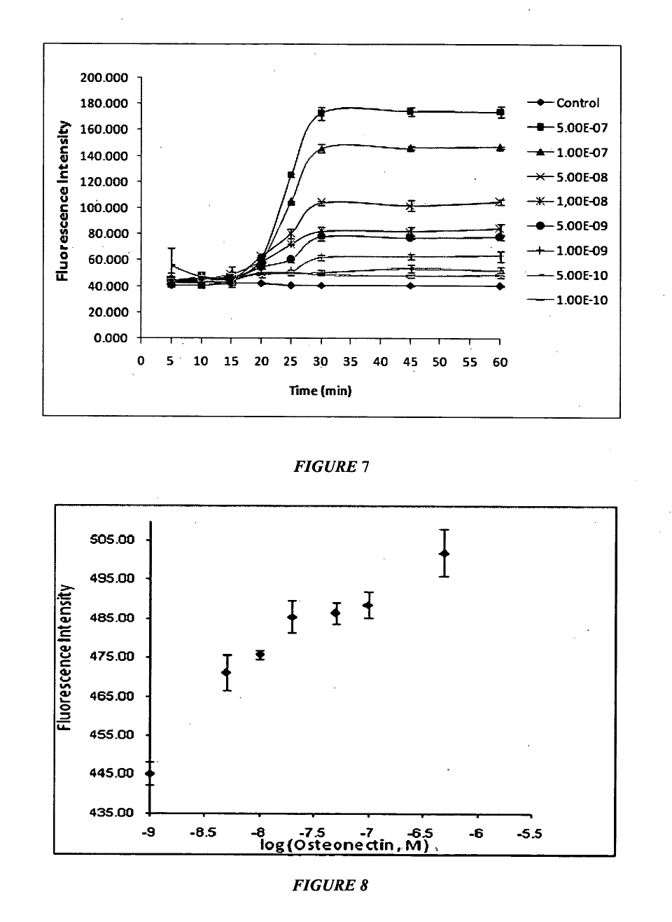

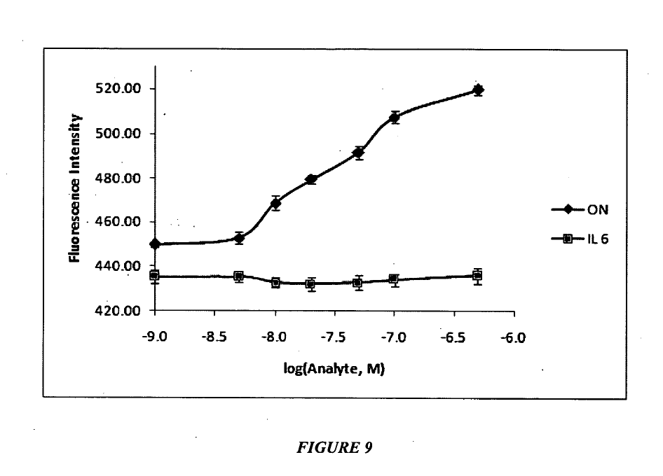

In this embodiment of the biosensor, a monoclonal antibody that selectively binds human bone osteonectin (FIG. 5) was covalently linked to a probe comprised of 2-Azido ATP labeled with the fluorophore ALEXA FLUOR® 594 cadaverine at the unconventional binding site (i.e., purine-binding site), located in the variable region between the heavy and light chains of the antibody. 2-azido ATP was selected over 8-azido ATP in this embodiment, as it was observed that 2-azido ATP labels this osteonectin antibody with a greater efficiency as compared to 8-azido ATP (FIG. 6). The antigen-binding site in the antibody is unaffected by this docking of the fluorophore-labeled probe. Upon binding to the analyte, conformational changes within the labeled antibody affects the fluorescence intensity of probe. The intensity of fluorescence of the label was found to increase with the increase of the osteonectin concentration in the sample (FIGS. 7 and 8). FIG. 9 shows that the change in fluorescence was specific to the concentration of osteonectin, as an increase in fluorescence in the presence of an unrelated molecule (IL-6) did not occur.

### Example 3

The present Example sets forth a general protocol for development of a biosensor disclosed herein, which incorporates any desired antibody. The antibody portion of the biosensor can be selected based on its capabilities to selectively bind to an antigen-binding site on the antibody a desired molecule of interest. The probe portion of the biosensor can comprise a purine molecule (e.g., ATP or analog thereof) that binds to a purine-binding site on the antibody and any label (e.g., a fluorophore), so long as the label emits a detectable signal that differs upon a conformational change in the antibody after binding of the antigen of interest, to thereby detect the molecule of interest. Thus, the present protocols can be used as a universal method for design of antibody-based biosensors that can detect any desired molecule of interest. The universal adaptability of the disclosed methods and sensors is based in part on two factors. First, the identification of unconventional purine-binding sites within the antibody that are conserved sequences across a wide variety of antibodies and are distinct from the antigen-binding sites, such that binding a probe to the site does not interfere with antigen binding by the antibodies. Second, the particular label is selected such that a detectable change in emitted signal by the antibody occurs upon a conformational change in the antibody resulting from binding of the molecule of interest to the antibody.

To demonstrate the conservation across different antibodies of the unconventional purine binding site (Rajagopalan et al., 1996), the sequences of a series of IgGs are aligned. Sequences used in the alignment were from antibodies whose structure have been solved. The sequences were obtained from the Protein Data Bank (PDB) by searching with the keyword “Fab antibody”. The search resulted in 549 hits (search conducted in December 2007). The entries were examined, and the first 20 non-redundant sequences, each containing a heavy chain and a light chain, were selected for the alignment. A sequence used for computer modeling in an earlier study, 1MRC, was also included. The PDB ID numbers corresponding to these sequences and their antigens are provided in Table 1.

TABLE 1
Sequences Used in the Antibody Alignment

| PDB ID | Antigen | Reference |
|---|---|---|
| CTS | Cortisol | Le Calvez et al., 1995 |
| 1MRC | RNA | Pokkuluri et al., 1994 |
| 1KEL | Catalytic antibody, Oxygenation | Hsieh-Wilson et al., 1996 |
| 1QBM | Cytochrome C | Mylvaganam et al., 1998 |
| 1MJ8 | Catalytic antibody, esterase | Ruzheinikov et al., 2003 |
| 2F58 | Neutralizing antibody | Stanfield et al., 1999 |
| 2A6I | Arsonate germline | Sethi et al., 2006 |
| 1EMT | Buckminsterfullerene | Braden et al., 2000 |
| 1S5I | Human Interleukin-2 | Pletnev et al., 2004 |
| 1RUK | Catalytic antibody, water-oxidation | Zhu et al., 2004 |
| 1CR9 | Prion | Kanyo et al., 1999 |
| 1BEY | CD52 (CAMPATH-1) therapeutic | Cheetham et al., 1998 |
| 1M71 | Shigella flexneri Y | Vyas et al., 2002 |
| | Lipopolysaccharide | |
| 1AIF | Idiotypic | Ban et al., 1995 |
| 1FGN | Human tissue | Huang et al., 1998 |
| 1A3R | Rhinovirus neutralizing antibody | Tormo et al., 1994 |
| 1C17 | HIV protease | Lescar et al., 1999 |
| 1FPT | Poliovirus neutralizing antibody | Wien et al., 1995 |
| 1KEG | dTT(6-4)TT | Yokoyama et al., 2000 |
| 1A5F | E-selectin 7A9 | Rodriguez-Romero et al., |
| | | 1998 |
| 2AJX | Cocaine catalytic antibody | Zhu et al., 2006 |
| 1R3I | Potassium transporter | Zhou et al., 2003 |

The heavy chains and light chains of the 22 sequences were separated and submitted to the online server of the T-Coffee software (O'Sullivan et al., 2004; http://tcoffee.vital-it.ch/cgi-bin/Tcoffee/tcoffeecgi/index.cgi) for alignment. Alignments of the fragments next to residues determined by computer modeling as binding the purine-based probe, for both the light and the heavy chain, are summarized in Table 2. The three residues that have been determined to make contact with the photolabel in the structure of 1MRC (Y36 from the light chain, D101 and W103 from the heavy chain) are highlighted. All three residues are highly conserved among the sequences, with W103 absolutely invariant.

TABLE 2
Sequence Alignment of Antibodies.⁺

(SEQ
ID NO:)

Light chain

| | |
|---|---|
| 36 | |
| CTS VITQSPSSLAVSAGERVTMTCRSSQSLFNSRIRKN-YLAWYQHKPGQSPKLLIYWAST | 1 |
| 1MRC VMTQTPLSLPVSLGDQASISCRSSQSLVHS-NG-NTYLHWYLQKPGQSPKLLIYKVSN | 2 |
| 1A3R VMTQSPSSLTVTTGEKVTMTCKSSQSLLNSRTQ-KNYLTWYQQKPGQSPKLLIYWAST | 3 |
| 1A5F VMTQSPSSLTVTTGEKVTMTCKSSQSLLNS-GAQKNYLTWYQQKPGQSPKLLIYWAST | 4 |
| 1KEG LMTQTPLSLPVSLGDQASISCRSSQSIVHS-NG-NTYLEWYLQKPGQSPKLLIYKVSN | 5 |
| 1M71 VLTQTPLSLPVRLGDQASISCRSSQSLLHS-DG-NTYLHWYLQKPGQSPKLLIYKVSN | 6 |
| 1CL7 LMTQTPLYLPVSLGDQASISCRSSQTIVHN-NG-NTYLEWYLQKPGQSPQLLIYKVSN | 7 |
| 1FPT VMTQTPLSLPVSLGDQASISCSSSQSLVHS-NG-KTYLHWYLQKPGQSPKLLIYKVSN | 8 |
| 1CR9 VMTQTPLSLSVTIGQPASISCKSSQSLLDS-DG-KTYLIWVFQRPGQSPKRLIFLVSK | 9 |
| 1S5I QMTQTPLTLSVTIGQPASISCESSQSLLYS-NG-KTYLNWLLQRPGQSPKRLIYLVSK | 10 |
| 1RUK VMTQSPKTISVTIGQPASISCKSSQRLLNS-NG-KTFLNWLLQRPGQSPKRLIYLGTK | 11 |
| 1KEL LMTQTPLSLPVSLGDQASISCRFSQSIVHS-NG-NTYLEWYLQKSGQSPKLLIYKVSN | 12 |
| 1MJ8 VMTQAAPSVPVTPGESVSISCRSSKSLLHS-NG-NTYLYWFLQRPGQSPQLLIYRMSN | 13 |
| 2AJX VITQDELSNPVTSGESVSISCRSSRSLLYK-DG-RTYLNWFLQRPGQSPQLLIYLMST | 14 |
| lAIF QLTQSPAFMAASPGEKVTITCSVSSSISSS N-----LHWYQQKSETSPKPWIYGTSN | 15 |
| 2F58 VLTQSPASLAVSLGQRATISCKASQGV-DF-DG-ASFMNWYQQKPGQPPKLLIFAAST | 16 |
| lEMT QMTQTTSSLSASLGDRVTFSCSASQDI-------SNYLNWYQQKPDGTIKLLIYYTSS | 17 |
| 2A61 QMTQTTSSLSASLGDRVTISCRASQDI-------SNYLNWYQQKPDGTVKLLIYYTSR | 18 |
| 1FGN KMTQSPSSMYASLGERVTITCKASQDI-------RKYLNWYQQKPWKSPKTLIYYATS | 19 |
| 1QBM QMTQSPASLSASVGETVTITCRASGNI-------HNYLAWYQQKQGKSPQLLVYNAKT | 20 |
| 1R31 LLTQSPAILSVSPGERVSFSCRASQSI-------GTDIHWYQQRTNGSPRLLIKYASE | 21 |
| 1BEY QMTQSPSSLSASVGDRVTITCKASQNI-------DKYLNWYQQKPGKAPKLLIYNTNN | 22 |
| :** . *: .:::* * : : *: *: . : : . | |

Heavy chain

| | |
|---|---|
| 101 103 | |
| CTS GSLRSEDTAIYFCARWAAY-------KHYFDYWGQGTALTVSSAKTTPPSVYPLAPGC | 23 |
| 1MRC SSLTSEDSAVYYCANLRGY----------FDYWGQGTTLTVSSAKTTPPSVYPLAPGC | 24 |
| 1A3R SSLTSEDTAVYYCDGYYS--------YYDMDYWGPGTSVTVSSAKTTAPSVYPLAPVC | 25 |
| 1CR9 SSLTSEDTAVYYCNAD------------LHDYWGQGTTLTVSSAKTTAPSVYPLAPVC | 26 |
| 1AIF NSLRAEDTGIYYCVLRPL-------FYYAVDYWGQGTSVTVSSAKTTPPSVYPLAPGS | 27 |
| 1M71 NNLRAEDTGIYYCTRGGA--------VGAMDYWGQGTSVTVSSATTTAPSVYPLVPGC | 28 |
| 1BEY SSVTAADTAVYYCAREGH-------TAAPFDYWGQGSLVTVSSASTKGPSVFPLAPSS | 29 |
| 1KEL NTLRAEDSATYYCARWGS---------YAMDYWGQGTSVTVSSAKTTPPSVYPLAPGS | 30 |
| 1RUK NSVTTEDTATYYCAGLLW-------YDGGAGSWGQGTLVTVSAAKTTAPSVYPLAPVC | 31 |
| 1S5I NSVTTEDTATYYCASYDD-------Y-TWFTYWGQGTLVTVSAAKTTPPSVYPLAPGS | 32 |
| 2AJX NSVTTEDTATYYCARYDY-------YGNTGDYWGQGTSVTVSSAKTTPPSVYPLAPGT | 33 |
| 2F58 NSVTTEDTATYYCAREEAMPYGNQAYYYAMDCWGQGTTVTVSSAKTTPPSVYPLAPGS | 34 |
| 1A5F SSLTSEDTAVYYCARVGLS------YWYAMDYWGQGTSVTVSSAKTTPPSVYPLAPGS | 35 |
| 1QBM SSLTSEDTAVYYCAGYD---------YGNFDYWGQGTTLTVSSAETTPPSVYPLAPGT | 36 |
| 1EMT SSLTSVDSAVYFCATSS-------------AYWGQGTLLTVSAAKTTPPSVYPLAPGS | 37 |
| 1FGN SSLTSEDTAVYYCARDNSY---------YFDYWGQGTTLTVSSAKTTPPSVYPLAPGS | 38 |
| 1R3I SSLTSEDSAVYYCARERG--------DGYFAVWGAGTTVTVSSAKTTPPSVYPLAPGS | 39 |
| 1FPT SSLTSDDSAVYFCARDFYD------YDVGFDYWGQGTTLTVSSAKTTAPSVYPLAPVC | 40 |
| 1MJ8 SSLTSDDSAVYYCARKHYF-------YDGVVYWGQGTLVTVSAAKTTAPSVYPLAPVC | 41 |
| 1KEG SSLTNEDSAVYYCTRRSGY------KYYALDYWGQGTSVTVSSAKTTAPSVYPLAPVC | 42 |
| 2A61 RSLTSEDSAVYFCARSVYY-----GGSYYFDYWGQGTTLTVSSAKTTPPSVYPLAPGS | 43 |
| 1CL7 NNLKNEDTATYFCVRDRHD------YGEIFTYWGQGTTVTVSSAKTTPPSVYPLAPGS | 44 |
| .: *:. *:* ** *: :***:* *. ***:**.* | |

⁺D101 and W103 in the heavy chain, and Y36 in the light chain of 1MCR and their counterpart in other sequences are highlighted. The marks under the sequences indicate invariant (*), highly conserved (:) and conserved (.) residues.

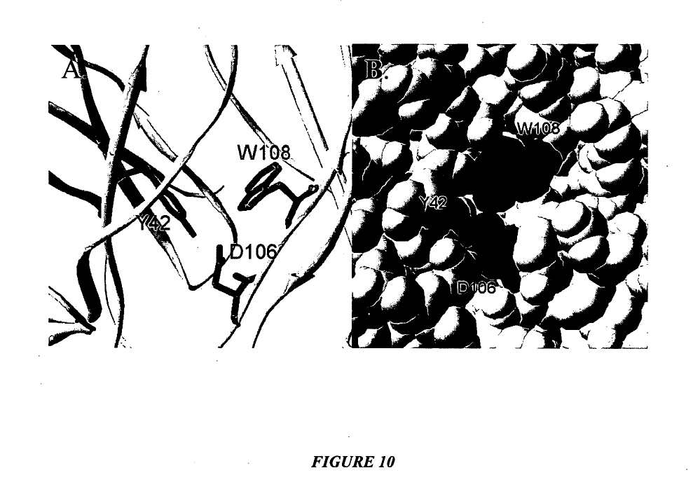

Cortisol was chosen as an exemplary biomarker for these biosensor development studies. As such, an anti-cortisol antibody (GenBank access code: AAB33872 for the heavy chain; AAB33873 for the light chain) was also included in the group of selected antibodies. The cortisol antibody is abbreviated as CTS in Tables 1 and 2. A structural model of the anti-cortisol antibody was generated with the ESyPred3D software using the crystal structure of the previously discussed anti-RNA antibody (1MRC) as the template. FIG. 10A is a ribbon diagram showing a close-up of the putative purine-binding site. The side chains of the three residues are shown: Y42 (light chain), D106 and W108 (heavy chain), which correspond to Y36, D101 and W103 in 1MRC, respectively. These residues are positioned within close distance, and capable of making contact simultaneously with the purine probe. The space-filled structure model (FIG. 10B) shows that the aromatic ring of W108 is not fully buried within the binding site. As a consequence, it may serve as a “sticky patch” when encountering the photolabel. Without wishing to be bound by theory, it is hypothesized that other residues in the vicinity of this “sticky patch” could form a binding pocket to fit the purine portion of the probe.

These data indicate that the purine-binding site is highly conserved across different antibodies. As such, a purine-based probe as disclosed herein can be incorporated into a biosensor comprised of any desired antibody. This provides for the development of a universal system of antibody-based biosensors.

As disclosed herein, it has now been determined that when the molecule of interest binds the antibody portion of the biosensor, it induces a conformational change within the antibody. The conformational change induces a change in the signal emitted by the label portion of the probe bound to the antibody. The change in signal is correlated with the binding, and therefore presence of, the molecule of interest. Thus, a label is selected based in part on it being capable of its emitted signal being detectably altered upon the conformational change in the antibody. Fluorophores are one example of labels that generally meet these criteria. For example, as demonstrated hereinabove, 2-azido-ATP can be a very efficient label, in some embodiments performing even better than 8-azido-ATP. However, both labels can be utilized with the biosensors disclosed herein.

In some embodiments, fluorescent derivatives of 2-azido-ATP or 8-azido-ATP can be prepared and employed for coupling to the antibody portion of the biosensor by photolabeling and attachment at the purine-binding site of the antibodies. The recognition by the antibody of its antigen (molecule of interest) in conjunction with the change of environment that the fluorescent probe experiences upon the conformational change constitutes the basis for the universal reagentless biosensors disclosed herein.

In particular embodiments, 2-N₃-ATP (i.e., 2-azido-ATP) can be conjugated to the fluorophore ALEXA FLUOR® 594 cadaverine using 1-ethyl-3-(3-dimethylaminopropyl) carbodiimide and N-hydroxysulfosuccinimide coupling chemistry to generate the probe portion of the biosensor. The probe (Formula I), can be purified using a DEAE (diethylaminoethyl) cellulose column and triethylammonium carbonate (0-300 mM) as elution gradient. The purified probe can then be covalently-bound to the selected antibody at the purine-binding site via the azido group of the probe by photolytic reaction, as disclosed in Example 1.

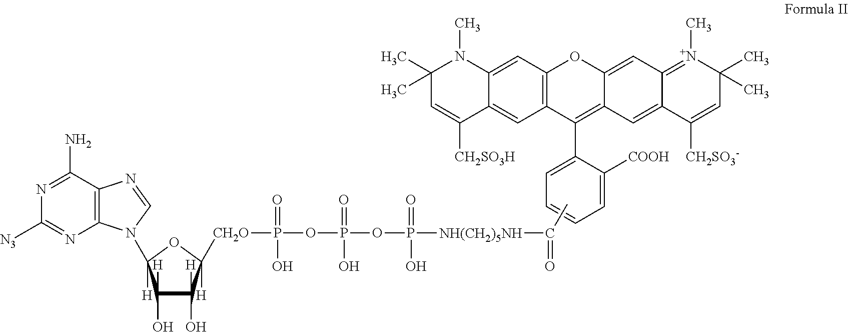

Once a biosensor is produced, it can be analyzed to determine if it accurately binds a molecule of interest and emits a signal correlating to the binding. For each biosensor, it can be established whether binding of the label-conjugated purine probe to the unconventional site of the antibody affects the binding of the targeted biomarker. If desired, it can further be determined whether binding of the biomarker to the antibody labeled with the label conjugated purine can cause a conformational change of the antibody, which can alter the signal intensity of the label. The change in the signal intensity of the probe can be utilized to monitor the concentration of biomarker in the samples. This approach provides for the development of a highly sensitive, selective, easy to use, and reagentless biosensing system, which can be based on fluorescence. As shown in Examples 1 and 2, it has now been verified that a fluorophore-conjugated ATP label can bind to the unconventional site of antibodies without affecting the antigen binding to the antibody. Thus, the change in the fluorescence intensity of the label can be correlated to the concentration of biomarker in the sample.

The conditions for the labeling of the antibody with the purine-based probe (such as pH during conjugation, selected buffer, etc.) can be optimized so that labeled antibody reagents are obtained that lead to assays with detection limits that match the concentrations expected in the samples. Additionally, if desired, the binding constant (K_{D}) of the antibody to the biomarker can be determined, as well as measuring the corresponding association and dissociation rate constants using, for example, microcalorimetry and/or surface plasmon resonance (SPR, Biacore). Finally, the response characteristics of the reagentless sensing system can be optimized in terms of selectivity, sensitivity and response time. Antibodies from more than one manufacturer can be evaluated to obtain biomarker assays with the best selectivity/sensitivity.

### REFERENCES

Ban, N.; Escobar, C.; Hasel, K. W.; Day, J.; Greenwood, A.; McPherson, A. “Structure of an anti-idiotypic Fab against feline peritonitis virus-neutralizing antibody and a comparison with the complexed Fab”, FASEB J 1995, 9, 107-114.
Barkenhoff, J. P. J., Hart, M. H., Aarden, L. A. J. Immunol. 143, 1175-1179 (1989).
Braden, B. C.; Goldbaum, F. A.; Chen, B. X.; Kirschner, A. N.; Wilson, S. R.; Erlanger, B. F. “X-ray crystal structure of an anti-Buckminsterfullerene antibody fab fragment: biomolecular recognition of C(60)”, Proc Natl Acad Sci USA 2000, 97, 12193-12197.
Brakenhoff, J. P. J., de Groot, E. R., Evers, R. F., Pannekoek, H., Aarden, L. A. J. Immunol. 39, 4116-4121 (1987).
Cheetham, G. M.; Hale, G.; Waldmann, H.; Bloomer, A. C. “Crystal structures of a rat anti-CD52 (CAMPATH-1) therapeutic antibody Fab fragment and its humanized counterpart”, J Mol Biol 1998, 284, 85-99.
Haley, B. E. Methods Enzymol. 20, 4771-487 (1991).
Hirano, T. et al. Nature 324, 73-76 (1986).
Hsieh-Wilson, L. C.; Schultz, P. G.; Stevens, R. C. “Insights into antibody catalysis: structure of an oxygenation catalyst at 1.9-angstrom resolution”, Proc Natl Acad Sci USA 1996, 93, 5363-5367.
Huang, M.; Syed, R.; Stura, E. A.; Stone, M. J.; Stefanko, R. S.; Ruf, W.; Edgington, T. S.; Wilson, I. A. “The mechanism of an inhibitory antibody on TF-initiated blood coagulation revealed by the crystal structures of human tissue factor, Fab 5G9 and TF.G9 complex”, J Mol Biol 1998, 275, 873-894.
Jones, S. A., Horiuchi, S., Topley, N., Yamamoto, N., Fuller, G. M. The FASEB Journal 15, 43-58 (2001).
Kanyo, Z. F.; Pan, K. M.; Williamson, R. A.; Burton, D. R.; Prusiner, S. B.; Fletterick, R. J.; Cohen, F. E. “Antibody binding defines a structure for an epitope that participates in the PrPC—>PrPSc conformational change”, J Mol Biol 1999, 293, 855-863.
Lescar, J.; Brynda, J.; Rezacova, P.; Stouracova, R.; Riottot, M. M.; Chitarra, V.; Fabry, M.; Horejsi, M.; Sedlacek, J.; Bentley, G. A. “Inhibition of the HIV-1 and HIV-2 proteases by a monoclonal antibody”, Protein Sci. 1999, 8, 2686-2696.
Luc, G. et al. Asteriscler. Thromb. Vasc. Biol. 23, 1255-1261 (2003).
Mendoza, C. E., Rosado, M. F., Bernal, L. Tex. Heart Inst. J. 28, 3-7 (2001).
Mylvaganam, S. E.; Paterson, Y.; Getzoff, E. D. “Structural basis for the binding of an anti-cytochrome c antibody to its antigen: crystal structures of FabE8-cytochrome c complex to 1.8 A resolution and FabE8 to 2.26 Å resolution”, J Mol Biol 1998, 281, 301-322.
O'Sullivan, O.; Suhre, K.; Abergel, c.; Higgins, D. G.; Notredame, C. “3DCoffee: combining protein sequences and structures within multiple sequence alignments”, J Mol Biol 2004, 340, 385-395.
Pavlinkova, G., Rajgopalan, K., Muller, S., Chavan, A., Sievert, G., Lou, D., O'Toole, C., Haley, B., Kohler, H. J. of Immunological Methods 201, 77-88 (1997).
Pletnev, V. Z.; Goryacheva, E. A.; Tsygannik, I. N.; Nesmeyanov, V. A.; Pletnev, S. V.; Pangborn, W.; Duax, W. “A New Crystal Form of the Fab Fragment of a Monoclonal Antibody to Human Interleukin-2: The Three-Dimensional Structure at 2.7 Å Resolution”, Russ J Bioorganic Chem 2004, 30, 417-420.
Pokkuluri, P. R.; Bouthillier, F.; Li, Y.; Kuderova, A.; Lee, J.; Cygler, M. “Preparation, characterization and crystallization of an antibody Fab fragment that recognizes RNA. Crystal structures of native Fab and three Fab-mononucleotide complexes”, J Mol Biol 1994, 243, 283-297.
Rajgopalan, K., Pavlinkova, G., Levy, S., Pokkuluri, P. R., Schiffer, M., Haley, B., Kohler, H. Proc. Natl. Acad. Sci. USA 93, 6019-6024 (1996).
Rodriguez-Romero, A.; Almog, O.; Tordova, M.; Randhawa, Z.; Gilliland, G. L. “Primary and tertiary structures of the Fab fragment of a monoclonal anti-E-selectin 7A9 antibody that inhibits neutrophil attachment to endothelial cells”, J Biol Chem 1998, 273, 11770-11775.
Ruzheinikov, S. N.; Muranova, T. A.; Sedelnikova, S. E.; Partridge, L. J.; Blackburn, G. M.; Murray, I. A.; Kakinuma, H.; Takashi, N.; Shimazaki, K.; Sun, J.; Nishi, Y.; Rice, D. W. “High-resolution crystal structure of the Fab-fragments of a family of mouse catalytic antibodies with esterase activity”, J Mol Biol 2003, 332, 423-435.
Sethi, D. K.; Agarwal, A.; Manivel, V.; Rao, K. V.; Salunke, D. M. “Differential epitope positioning within the germline antibody paratope enhances promiscuity in the primary immune response”, Immunity 2006, 24, 429-438.
Stanfield, R.; Cabezas, E.; Satterthwait, A.; Stura, E.; Profy, A.; Wilson, I. “Dual conformations for the HIV-1 gp120 V3 loop in complexes with different neutralizing fabs”, Structure Fold Des 1999, 7, 131-142.
Tormo, J.; Blaas, D.; Parry, N. R.; Rowlands, D.; Stuart, D.; Fita, I. “Crystal structure of a human rhinovirus neutralizing antibody complexed with a peptide derived from viral capsid protein VP2”, EMBO J. 1994, 13, 2247-2256.
Volpato, S., Guralnik. J. M., Ferrucci, L., Balfour, J., Chaves, P., Fried. L. P., Harris, T. B. Circulation 103, 947-953 (2001).
Vyas, N. K.; Vyas, M. N.; Chervenak, M. C.; Johnson, M. A.; Pinto, B. M.; Bundle, D. R.; Quiocho, F. A. “Molecular recognition of oligosaccharide epitopes by a monoclonal Fab specific for Shigella flexneri Y lipopolysaccharide: X-ray structures and thermodynamics”, Biochemistry 2002, 41, 13575-13586.
Wien, M. W.; Filman, D. J.; Stura, E. A.; Guillot, S.; Delpeyroux, F.; Crainic, R.; Hogle, J. M. “Structure of the complex between the Fab fragment of a neutralizing antibody for type 1 poliovirus and its viral epitope”, Nat Struct Bio/1995, 2, 232-243.
Yokoyama, H.; Mizutani, R.; Satow, Y.; Sato, K.; Komatsu, Y.; Ohtsuka, E.; Nikaido, O. “Crystal structure of the 64M-2 antibody Fab fragment in complex with a DNA dT(6-4)T photoproduct formed by ultraviolet radiation”, J Mol Biol, 2000, 299, 711-723.
Zhou, Y.; MacKinnon, R. “The occupancy of ions in the K+ selectivity filter: Charge balance and coupling of ion binding to a protein conformational change underlie high conduction rates”, J Mol Biol 2003, 333, 965-975.
Zhu, X.; Wentworth Jr., P.; Wentworth, A. D.; Eschenmoser, A.; Lerner, R. A.; Wilson, I. A. “Probing the antibody-catalyzed water-oxidation pathway at atomic resolution”, Proc Natl Acad Sci USA, 2004, 110, 2247-2252.
Zhu, X.; Dickerson, T. J.; Rogers, C. J.; Kaufmann, G. F.; Mee, J. M.; McKenzie, K. M.; Janda, K. D.; Wilson, I. A. “Complete reaction cycle of a cocaine catalytic antibody at atomic resolution”, Structure 2006, 14, 205-216.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A biosensor for detecting a molecule of interest, comprising:
(a) a probe, including a purine molecule and a fluorophore bound to the purine molecule; and
(b) an antibody, including an antigen-binding site that selectively binds the molecule of interest and a purine-binding site, wherein the probe is covalently bound at the purine molecule to the purine-binding site,
wherein binding of the molecule of interest to the antibody causes a conformational change in the antibody, which detectably alters a fluorescence intensity of the fluorophore such that the molecule of interest can be detected.

2. The biosensor of claim 1, wherein the purine molecule is a nucleotide or a nucleoside.

3. The biosensor of claim 1, wherein the purine molecule is an adenine or a guanine.

4. The biosensor of claim 3, wherein the purine molecule is an ATP analog or a GTP analog.

5. The biosensor of claim 4, wherein the purine molecule is 2-azido ATP or 8-azido ATP.

6. The biosensor of claim 1, wherein the fluorophore is an Alexa Fluor dye.

7. The biosensor of claim 6, wherein the probe is a compound of Formula II:

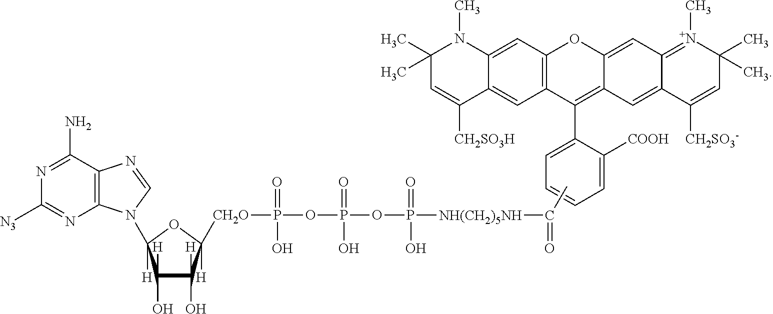

8. The biosensor of claim 1, wherein the antibody is a monoclonal antibody.

9. The biosensor of claim 1, wherein the purine-binding site is located within the variable domain of the antibody and at a location distinct from that of the antigen-binding site.

10. The biosensor of claim 9, wherein the purine-binding site comprises invariant amino acid residues within the variable domain.

11. The biosensor of claim 1, wherein the molecule of interest is Interleukin 6 (IL-6) or osteonectin.

12. The biosensor of claim 1, wherein the fluorescence intensity can be correlated to the concentration of the molecule of interest in a sample.

13. A system for continuous in vivo detection of a molecule of interest in a body of a subject, comprising:
(a) a detection and data collection device; and
(b) a biosensor capable of recognizing the molecule of interest, the biosensor being operably connected with the data and collection device and including:
i. a probe having a purine molecule and a fluorophore bound to the purine molecule; and
ii. an antibody having an antigen-binding site that selectively binds the molecule of interest and a purine-binding site, wherein the probe is covalently bound at the purine molecule to the purine-binding site,
wherein binding of the molecule of interest to the antibody causes a conformational change in the antibody, which detectably alters a fluorescence intensity of the fluorophore such that the molecule of interest can be detected.

14. The system of claim 13, wherein the purine molecule is a nucleotide or a nucleoside.

15. The system of claim 14, wherein the purine molecule is an adenine or a guanine.

16. The system of claim 15, wherein the purine molecule is an ATP analog or a GTP analog.

17. The system of claim 16, wherein the purine molecule is 2-azido ATP or 8-azido ATP.

18. The system of claim 13, wherein the fluorophore is an Alexa Fluor dye.

19. The system of claim 18, wherein the probe is a compound of Formula II:

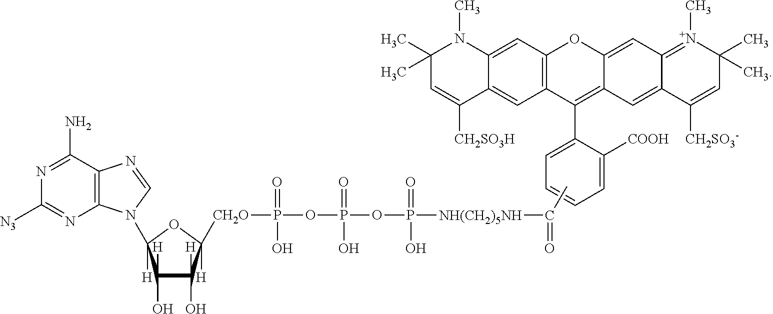

20. The system of claim 13, wherein the antibody is a monoclonal antibody.

21. The system of claim 13, wherein the purine-binding site is located within the variable domain of the antibody and at a location distinct from that of the antigen-binding site.

22. The system of claim 21, wherein the purine-binding site comprises invariant amino acid residues within the variable domain.

23. The system of claim 13, wherein the molecule of interest is Interleukin 6 (IL-6) or osteonectin.

24. The system of claim 13, wherein the fluorescence intensity can be correlated to the concentration of the molecule of interest in a sample.

25. A method for detecting a molecule of interest, comprising:
(a) contacting a biosensor with a sample comprising the molecule of interest, the biosensor including:
i. a probe, including a purine molecule and a fluorophore bound to the purine molecule; and
ii. an antibody, including an antigen-binding site that selectively binds the molecule of interest and a purine-binding site, wherein the probe is covalently bound at the purine molecule to the purine-binding site;
(b) binding the molecule of interest to the antibody, thereby resulting in a conformational change in the antibody, which detectably alters a fluorescence intensity of the fluorophore;
(c) detecting as a signal the altered fluorescence intensity of the fluorophore; and
(d) collecting and displaying the signal with a detection and data collection device, to thereby detect the molecule of interest.

26. The method of claim 25, wherein the molecule of interest is continuously detected.

27. The method of claim 25, wherein the molecule of interest is within an in vitro sample.

28. The method of claim 25, wherein the molecule of interest is within a blood stream in a body of a subject.

29. The method of claim 25, wherein the biosensor is implanted in a body of a subject.

30. The method of claim 25, wherein the purine molecule is a nucleotide or a nucleoside.

31. The method of claim 30, wherein the purine molecule is an adenine or a guanine.

32. The method of claim 31, wherein the purine molecule is an ATP analog or a GTP analog.

33. The method of claim 32, wherein the purine molecule is 2-azido ATP or 8-azido ATP.

34. The method of claim 25, wherein the fluorophore is an Alexa Fluor dye.

35. The method of claim 34, wherein the probe is a compound of Formula II:

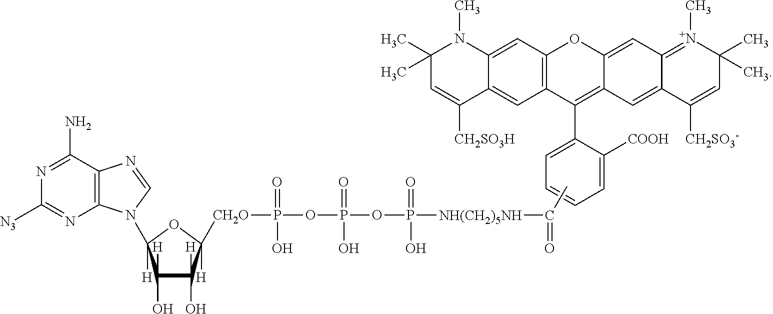

36. The method of claim 25, wherein the antibody is a monoclonal antibody.

37. The method of claim 25, wherein the purine-binding site is located within the variable domain of the antibody and at a location distinct from that of the antigen-binding site.

38. The method of claim 37, wherein the purine-binding site comprises invariant amino acid residues within the variable domain.

39. The method of claim 25, wherein the molecule of interest is Interleukin 6 (IL-6) or osteonectin.

40. The method of claim 25, wherein the fluorescence intensity correlates to the concentration of the molecule of interest in a sample.
